(19) United States
(12) Patent Application Publication
(10) Pub. No.: US 2005/0267422 A1
(43) Pub. Date: Dec. 1, 2005

(54) Fluid delivery apparatus

(76) Inventor: Marshall S. Kriesel, St. Paul, MN (US)

Correspondence Address: JAMES E. BRUNTON, ESQ., P. O. BOX 29000, GLENDALE, CA, 91209, US

(21) Appl. No.: 10/855,478

(22) Filed: May 26, 2004

(51) Int. Cl. A61M 31/00

(52) U.S. Cl. 604/288.01

(57) ABSTRACT

A fluid delivery device having a self-contained, precision mechanical spring type stored energy source for expelling fluids at a precisely controlled rate. The device can be used by lay persons in a non-hospital environment for the precise infusion of pharmaceutical fluids, such as insulin and the like, into an ambulatory patient at controlled rates over extended periods of time. In one form of the apparatus of the invention, there is provided a unique, microchannel type rate control assembly that is disposed intermediate the fluid reservoir outlet and the outlet port of the device.

### BACKGROUND OF THE INVENTION

1. Field of the Invention

The present invention relates generally to fluid delivery devices. More particularly, the invention concerns an improved apparatus for infusing medicinal agents into an ambulatory patient at specific rates over extended periods of time.

2. Discussion of the Invention

Many medicinal agents require an intravenous route for administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by mediating its therapeutic effectiveness. Certain classes of new pharmacologic agents possess a very narrow range of therapeutic effectiveness, for instance, too small a dose results in no effect, while too great a dose results in toxic reaction.

In the past, prolonged infusion of fluids has generally been accomplished using gravity flow methods, which typically involve the use of intravenous administration sets and the familiar bottle suspended above the patient. Such methods are cumbersome, imprecise and require bed confinement of the patient. Periodic monitoring of the apparatus by the nurse or doctor is required to detect malfunctions of the infusion apparatus.

A variety of fluid delivery devices from which fluids are controllably expelled by stored energy means provided in the form elastomeric film materials have been devised by the present inventor. The elastomeric film materials used in these devices as well as various alternate constructions of such devices are described in detail in U.S. Pat. No. 5,205,820 issued to the present inventor. A low-profile fluid delivery apparatus invented by the present inventor is described in U.S. Pat. No. 5,716,343.

Devices from which liquid is expelled from a relatively thick-walled bladder by internal stresses within the distended bladder have also been suggested in the past. Such bladder, or “balloon” type, devices are described in U.S. Pat. No. 3,469,578 issued to Bierman and in U.S. Pat. No. 4,318,400, issued to Perry. The devices of the aforementioned patents also disclose the use of fluid flow restrictor's external of the bladder for regulating the rate of fluid flow from the bladder.

The prior art bladder type infusion devices are not without drawbacks. Generally, because of the very nature of bladder or “balloon” configuration, the devices are unwieldy and are difficult and expensive to manufacture and use. Further, the devices are somewhat unreliable and their fluid discharge rates are frequently imprecise.

The apparatus of the present invention overcomes many of the drawbacks of the prior art by eliminating the bladder and also eliminating the elastomeric film energy source and making use of recently developed, high precision mechanical springs which function in cooperation with an expandable bellows assembly as an internal stored energy source for controllably forcing fluid from the apparatus reservoir.

The apparatus of the present invention can be used with minimal professional assistance in an alternate health care environment, such as the home. By way of example, devices of the invention can be comfortably and conveniently removably affixed to the patient's body or to the patient's clothing and can be used for the continuous infusion of antibiotics, hormones, steroids, blood clotting agents, analgesics, and like medicinal agents. Similarly, the devices can be used for I-V chemotherapy and can accurately deliver fluids to the patient in precisely the correct quantities and at extended microfusion rates over time.

As will be better understood from the description which follows, the inventions described herein are directed toward providing novel fluid delivery devices which are low profile and are eminently capable of meeting the most stringent of fluid delivery tolerance requirements. In this regard, medical and pharmacological research continues to reveal the importance of the manner in which a medicinal agent is administered. The delivery device, while not an active pharmacological agent, may enhance the activity of the drug by mediating its therapeutic effectiveness. For example, certain classes of pharmacological agents possess a very narrow dosage range of therapeutic effectiveness, in which case too small a dose will have no effect, while too great a dose can result in toxic reaction. In other instances, some forms of medication require an extended delivery time to achieve the utmost effectiveness of a medicinal therapeutic regimen.

By way of example, the therapeutic regimens used by insulin-dependent diabetics provide a good example of the benefits of carefully selected delivery means. The therapeutic object for diabetics is to consistently maintain blood glucose levels within a normal range. Conventional therapy involves injecting insulin by syringe several times a day, often coinciding with meals. The dose must be calculated based on glucose levels present in the blood. If the dosage is off, the bolus administered may lead to acute levels of either glucose or insulin resulting in complications, including unconsciousness or coma. Over time, high concentrations of glucose in the blood can also lead to a variety of chronic health problems, such as vision loss, kidney failure, heart disease, nerve damage, and amputations.

A recently completed study sponsored by the National Institutes of Health (NIH) investigated the effects of different therapeutic regimens on the health outcomes of insulin dependent diabetics. This study revealed some distinct advantages in the adoption of certain therapeutic regimens. Intensive therapy that involved intensive blood glucose monitoring and more frequent administration of insulin by conventional means, i.e., syringes, throughout the day saw dramatic decreases in the incidence of debilitating complications.

In those embodiments of the invention described in U.S. Pat. No. 5,205,820 issued to the present inventor, the fluid delivery apparatus components generally included: a base assembly; an elastomeric membrane serving as a stored energy means; fluid flow channels for filling and delivery; flow control means; a cover; and an ullage, which comprised a part of the base assembly. The ullage in these devices typically comprises a semi-rigid structure having flow channels leading from the top of the structure through the base to inlet or outlet ports of the device.

In the rigid ullage configuration, the stored energy means of the device must be superimposed over the ullage to form the fluid-containing reservoir from which fluids are expelled at a controlled rate by the elastomeric membrane of the stored energy means tending to return to a less distended configuration in a direction toward the ullage.

Elastomeric membrane materials suitable for use as the stored energy means must possess certain physical characteristics in order to meet the performance requirements for a fluid delivery apparatus. More particularly, for good performance, the elastomeric membrane material must have good memory characteristics under conditions of high extension; good resistance to chemical and radiological degradation; and appropriate gas permeation characteristics depending upon the end application to be made of the device.

Once an elastomeric membrane material is chosen that will optimally meet the desired performance requirements, there still remain certain limitations to the level of refinement of the delivery tolerances that can be achieved using the rigid ullage configuration. These result primarily from the inability of the rigid ullage to conform to the shape of the elastomeric membrane near the end of the delivery period. This nonconformity can lead to extended delivery rate tail-off and higher residual problems when extremely accurate delivery is required. For example, when larger volumes of fluid are to be delivered, the tail-off volume represents a smaller portion of the fluid amount delivered and therefore exhibits much less effect on the total fluid delivery profile, but in very small dosages, the tail-off volume becomes a larger portion of the total volume. This sometimes places severe physical limits on the range of delivery profiles that may easily be accommodated using the rigid ullage configuration.

As will be better appreciated from the discussion which follows, the apparatus of the present invention by using precision mechanical springs overcomes many of the drawbacks found an elastomeric membrane type devices and provides a unique and novel improvement for a disposable dispenser of simple but highly reliable construction that may be adapted to many applications of use.

### SUMMARY OF THE INVENTION

It is an object of the present invention to provide a fluid delivery device having a self-contained, precision mechanical spring stored energy source for expelling fluids at a precisely controlled rate which is of a compact, low profile construction. More particularly, it is an object of the invention to provide such a device which can which can conveniently be used for the precise infusion of pharmaceutical fluids, such as insulin and the like, into an ambulatory patient at controlled rates over extended periods of time.

It is another object of the invention to provide an apparatus of the aforementioned character which small, compact, highly reliable and easy-to-use by lay persons in a non-hospital environment.

It is another object of the invention to provide an apparatus as described in the preceding paragraphs which can conveniently be used for intravenous infusion of fluids into an ambulatory patient.

A further object of the invention is to provide a low profile, fluid delivery device which can meet even the most stringent fluid delivery tolerance requirements. In this regard, in one form of the apparatus of the invention, there is provided a unique, microchannel type rate control assembly that is disposed intermediate the fluid reservoir outlet and the outlet port of the device.

Another object of the invention is to provide an apparatus of the class described which includes a fill assembly that can be conveniently used to controllably fill the fluid reservoir of the device.

Another object of the invention is to provide an apparatus of the character described which, due to its unique construction, can be manufactured inexpensively in large volume by automated machinery.

Other objects of the invention will become more apparent from the discussion which follows.

### BRIEF DESCRIPTION OF THE DRAWINGS

FIG. 1 is a generally perspective, rear view of one form of the fluid delivery device of the invention.

FIG. 2 is a generally perspective, front view of the fluid delivery device shown in FIG. 1.

FIG. 3 is a top plan view of the base component of the fluid delivery device of the invention.

FIG. 4 is a cross-sectional view taken along lines 4-4 of FIG. 3.

FIG. 5 is a bottom plan view of the base component.

FIG. 6 is enlarged cross-sectional view of the fluid delivery device shown in FIG. 2 of the drawings.

FIG. 7 is a cross-sectional view, similar to FIG. 6, but showing the fluid reservoir of the device in a filled condition

FIG. 8 is a cross-sectional, exploded view of the base assembly of the device shown in FIGS. 1 and 2.

FIG. 9 is a side elevational view of the rate control subassembly of the apparatus of the invention.

FIG. 10 is a view taken along lines 10-10 of FIG. 9.

FIG. 11 is a view taken along lines 11-11 of FIG. 9.

FIG. 12 is a view taken along lines 12-12 of FIG. 6.

FIG. 13 is a top plan view of an alternate form of finger spring assembly of the apparatus of the invention.

FIG. 14 is a side elevational view of the finger spring assembly shown in FIG. 13.

FIG. 15 is a top plan view of still another form of finger spring assembly of the apparatus of the invention.

FIG. 16 is a side elevational view of the finger spring assembly shown in FIG. 15.

FIGS. 17A, 17B, 17C, 17D and 17E when considered together comprise a generally diagramatical view of a number of alternate forms of springs and spring assemblies of the apparatus of the invention.

### DESCRIPTION OF THE INVENTION

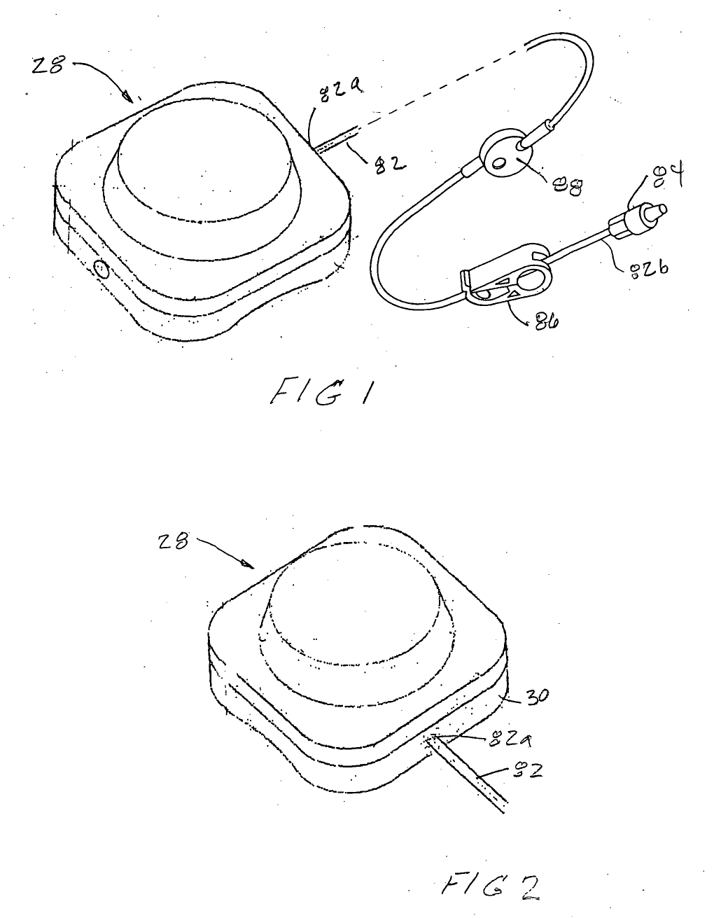

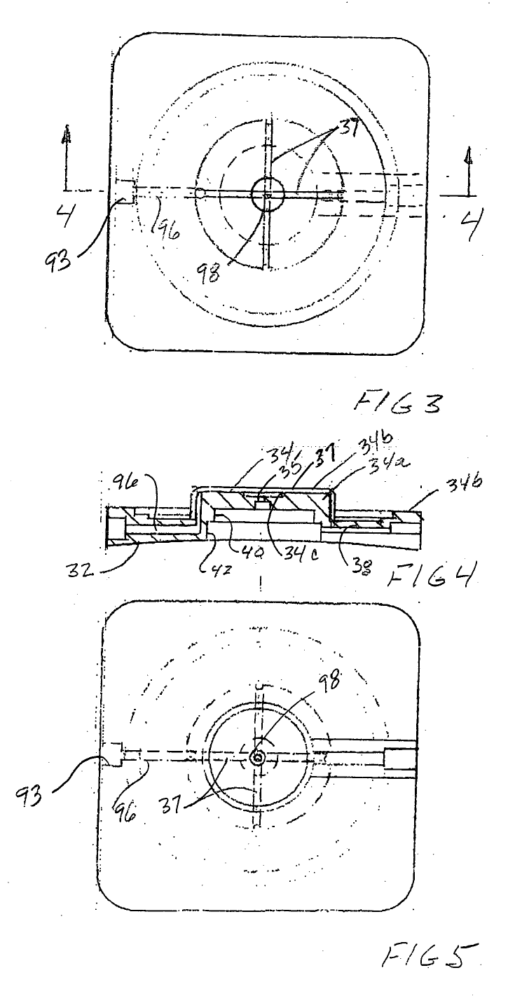

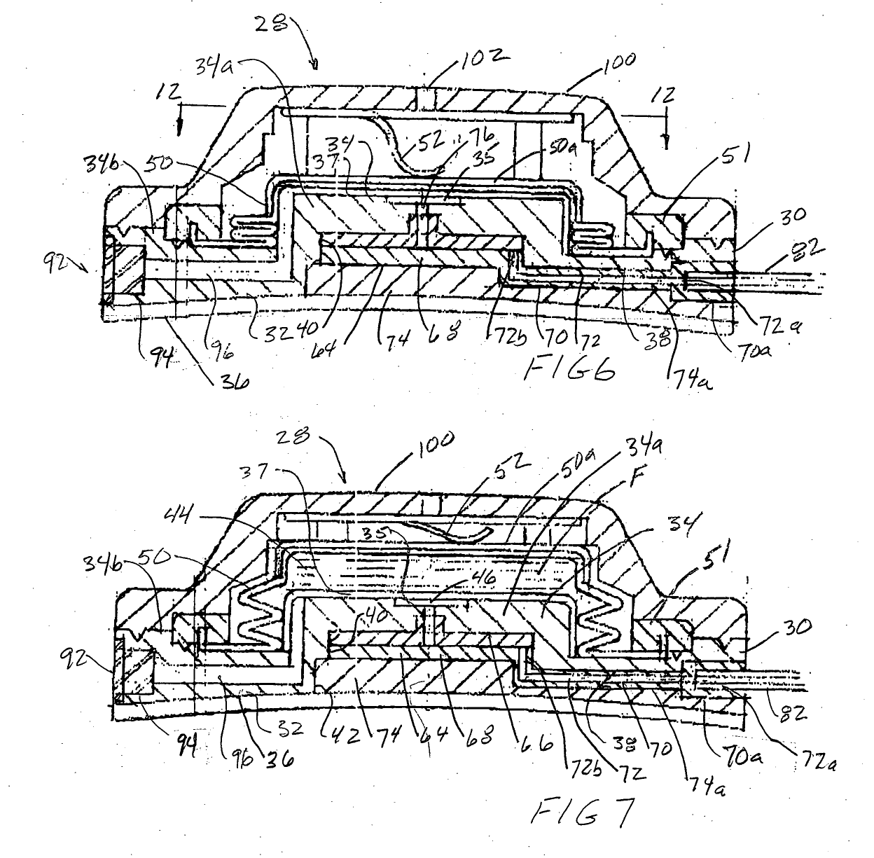

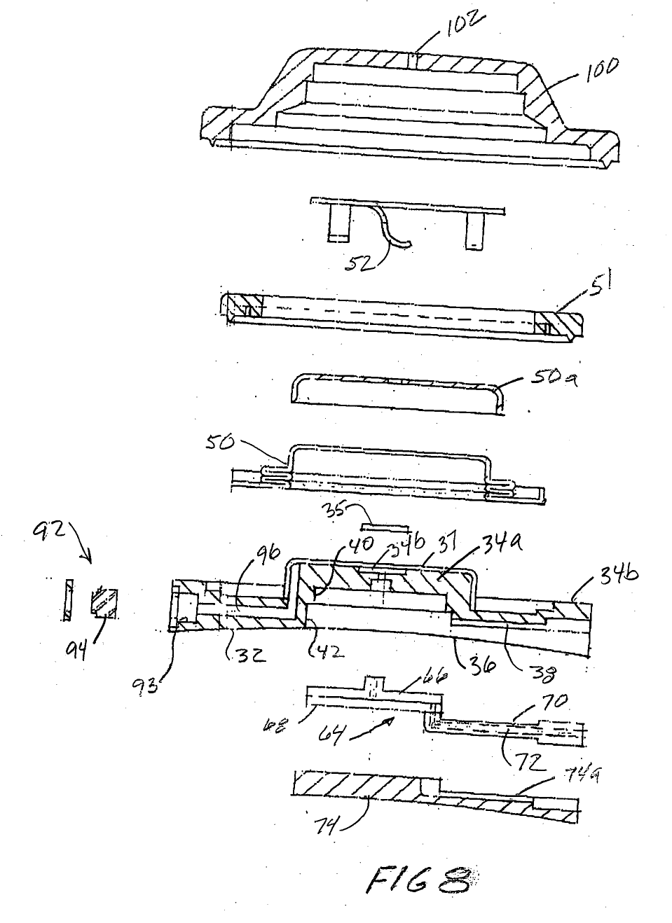

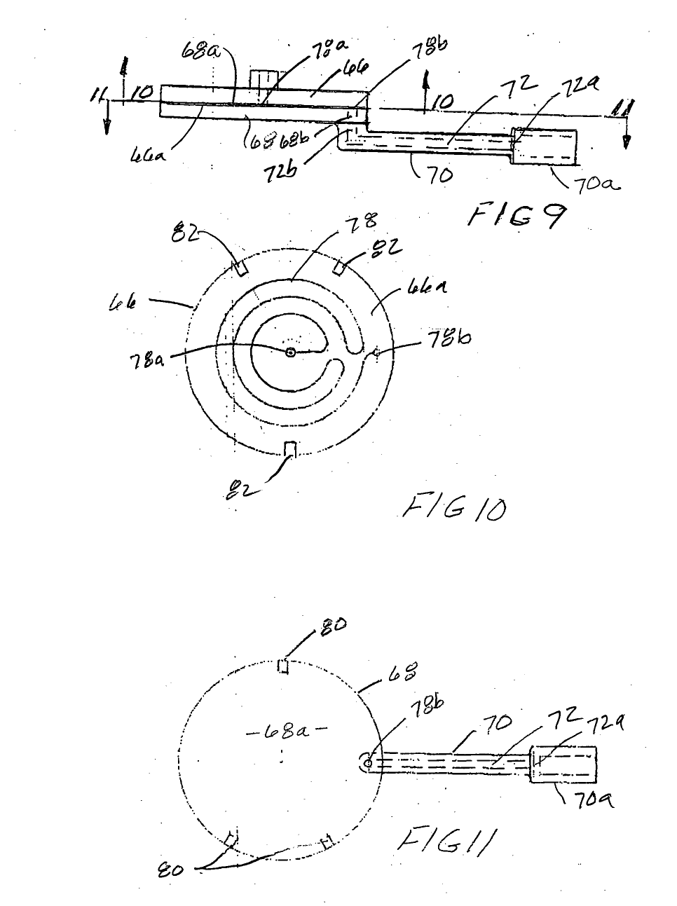

Referring to the drawings and particularly to FIGS. 1 through 7, one form of the device of the invention for use in intravenous infusion of medicinal fluid into a patient is there shown and generally designated by the numeral 28. As best seen by referring to FIGS. 6 and 7, the device here comprises a base assembly 30 which includes a base 32 having an upper surface 34, including a central portion 34a and peripheral portion 34b circumscribing central portion 34a (FIG. 4). As illustrated in FIGS. 3 and 8, central portion 34a is provided with a central counterbore 34c, which houses a filter 35 and is also provided with crossing, precisely formed fluid flow microchannels 37, the purpose of which will presently be described. Base 32 is provided with a lower surface 36 which is engageable with the patient when the device is taped or otherwise removably affixed to the patient. Formed within base 32 is a channel 38 and a pair of central counterbores 40 and 42 (FIGS. 4 and 7), the purpose of which will presently be described.

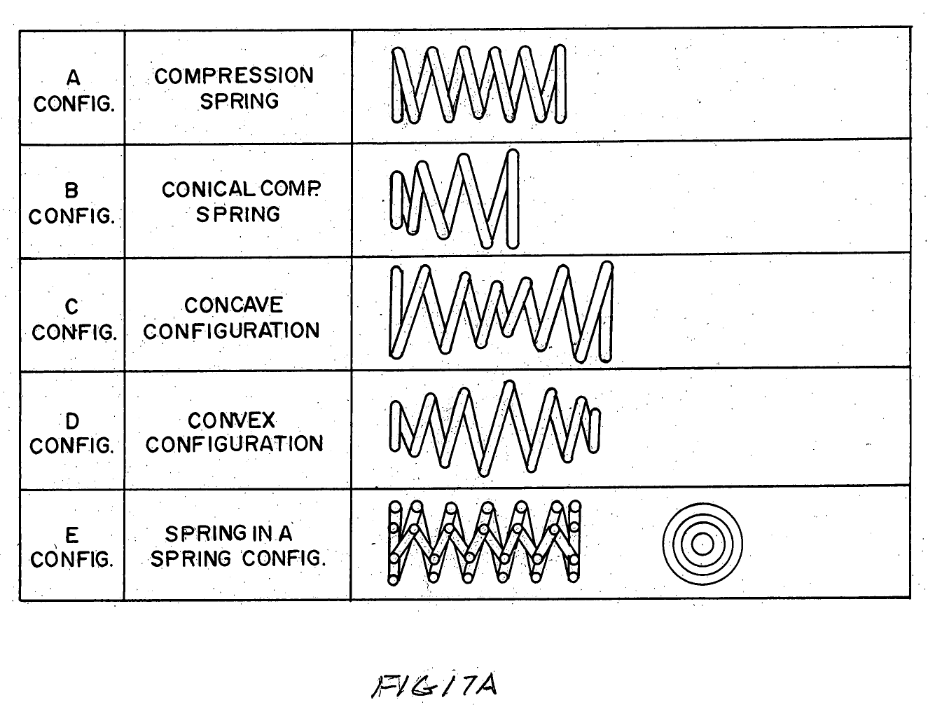

Forming an important aspect of the apparatus of the present invention is stored energy means for forming in conjunction with the central portion of 34a base 34 a reservoir 44 having an outlet 46 (FIG. 7). The stored energy means is here comprises an expandable bellows 50 which is superimposed over base 32 and is held and position by a capture ring 51. As illustrated in FIG. 7, the expandable bellows can be expanded from a first position shown and FIG. 6 to a second position shown in FIG. 7 as a result of pressure imparted by fluids “F” introduced into reservoir 44 via the fill means of the invention the character of which will presently be described. In the present form of the invention, the stored energy means further comprises a plurality of circumferentially spaced apart, yieldably deformable finger spring members 52 which are operably associated with bellows 50 (FIGS. 7 and 12). Each of the finger spring members 52 is yieldably deformed in the manner shown in FIG. 7 by movement of the expandable bellows toward the second position shown in FIG. 7. As the bellows 50 expands into the second position internal stresses are formed within the spring members, which forces tend to controllably return the expandable bellows to its first position. As the bellows moves toward its first position, fluid contained within reservoir 44 will be urged to flow outwardly of the reservoir through outlet 46 and toward the flow rate control means of the invention the character of which will next be described.

The important flow rate control means of the invention is here provided in the form of a rate control assembly 64 which includes a pair of generally circular shaped rate control plates 66 and 68 which are receivable within counterbore 40 formed in base 32. Rate control assembly 64 also includes a stem portion 70 which is connected to rate control plate 68 and which is provided with a fluid passageway 72 that has an inlet 72a and an outlet 72b. Stem portion 70 is partially received within a channel 38 formed in base 32 and, along with rate control plates 66 and 68, is held and position within base 32 by a base segment 74 which is provided with a groove 74a. Groove 74a partially receives stem portion 70 when the segment 74 is interconnected with base 32 in the manner shown in FIG. 6 of the drawings.

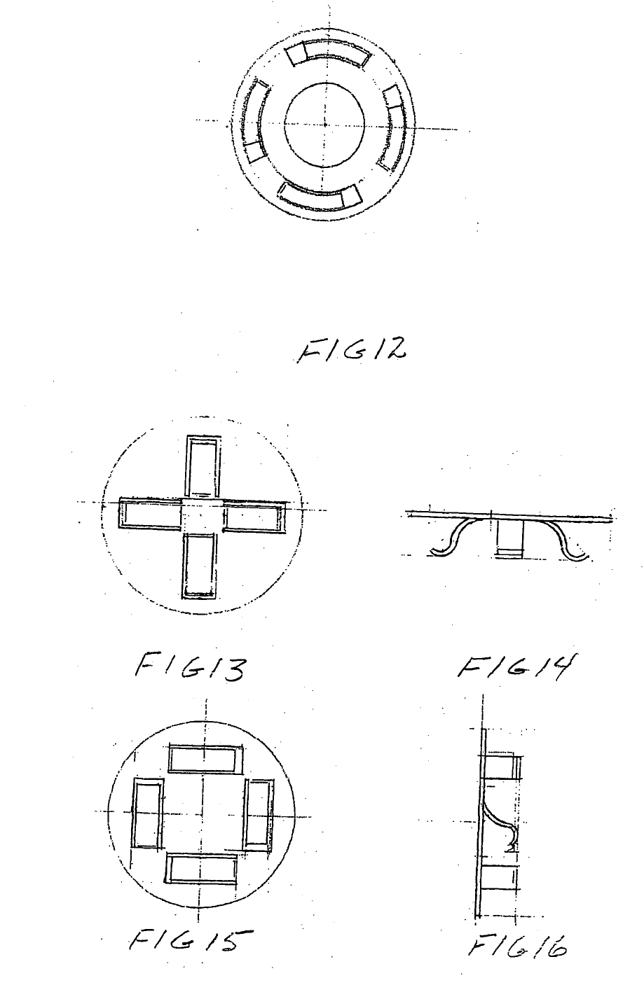

Turning particularly to FIGS. 9, 10 and 11, it is to be noted that the upper surface 68a of plate 68 is substantially planar and the lower surface 66a of plate 66, which is in mating engagement with upper surface 68a, is provided with a spiral shaped, laser-etched capillary or microchannel 78. Capillary 78 has an inlet port 78a that is in communication with reservoir 44 via a passageway 66b formed in plate 66 and an outlet port 78b that is in communication with inlet 72a of the passageway 72 formed an stem portion 70 via a passageway 68b formed in plate 68. Plates 66 and 68, which may be adhesively bonded together, are indexedly aligned by circumferentially spaced apart tabs 80 formed on plate 68 and circumferentially spaced apart slots 82 formed in plate 66 which closely receive tabs 80.

With the construction shown in the drawings, planar surface 68a of plate 68 cooperates with capillary 78 to form a fluid flow passageway through which fluid can controllably flow from reservoir 44 into the passageway 72 formed and stem 70. By controlling the length and depth of capillary 78, the rate of fluid flow flowing outwardly of outlet 78b can be precisely controlled. In this regard, it is to be understood that the capillary 78 of the flow rate control means can take several forms and be of various sizes depending upon the end use of the fluid delivery device.

The bonding material or adhesive used to bond together plates 66 and 68 may be of the thermo-melting variety or of the liquid or light curable variety. When thermo-melting adhesives are used, the adhesive material is melted into the two opposed surfaces, thereby interpenetrating these surfaces and creating a sealed channel structure. When liquid curable bonding materials, or adhesives, and light curable bonding materials are used, the adhesives may be applied to one of the surfaces of one of the plates. Subsequently, the other surface is brought into contact with the coated surface and the adhesive is cured by air exposure or via irradiation with a light source. Liquid curable bonding materials or adhesives may be elastomeric (e.g. thermoplastic elastomers, natural or synthetic rubbers, polyurethanes and silicones). Elastomeric bonding materials may or may not require pressure to seal the channel system. They may also provide closure and sealing to small irregularities in the opposed surface of the channel system.

It should also be understood that alternate bonding techniques such as sonic welding and laser thermal bonding techniques can also be used to bond together plates 66 and 68.

Connected to stem portion 70 of the rate control assembly 64 is the fluid delivery means of the invention. This latter mean comprises an elongated delivery line 82 having an inlet end 82a and an outlet end 82b. A conventional luer assembly 84 is affixed proximate outlet 82b, A line clamp 86 and a gas vent assembly 88, both of conventional construction, are disposed between the inlet and outlet ends of delivery line 82 (FIG. 1). As best seen in FIG. 6, the inlet end of the delivery line is telescopically received within an enlarged diameter portion 70a of stem portion 70 and is affixed thereto as by adhesive bonding.

Filling of reservoir 44 with a selected beneficial agent, or medicinal fluid, is accomplished by filling means which here comprises a septum assembly 92 which is connected to base 32 in the manner shown in FIGS. 6 and 7. Septum assembly 92 includes a pierceable septum 94 which is pierceable by the cannula of a conventional syringe (not shown). Communicating with the cavity 93, which holds septum 94, is a fluid flow passageway 96, which, in turn, communicates with one of the earlier described microchannels 37 that terminates in an outlet port 98 that communicates with inlet 46 of reservoir 44. With this construction, medicinal fluid can be introduced into reservoir 44 using a conventional syringe. Alternatively, the fill means can comprise a luer fitting or any other suitable fluid interconnection of a character well known to those skilled in the art by which fluid can be controllably introduced into reservoir 44 to cause expandable bellows 50 to move into its expanded configuration as shown in FIG. 7.

As best seen in FIGS. 6, 7 and 8, a cover 100 is superimposed over base assembly 30 and functions to enclose spring 52 and bellows 50. Cover 100 includes venting means comprising a vent port 102 formed in the upper wall of the cover for venting gases contained within cover 100 to atmosphere during the expansion of bellows 50.

During filling of reservoir 44, which is accomplished in the manner previously described, the fluid being introduced into the reservoir under pressure via septum 92 will cause bellows 50 to move into the expanded configuration shown in FIG. 7. As the bellows is thus distended, a cover 50a, which covers bellows 50 (FIG. 8), will engage the yieldably deformable finger spring members 52 causing the fingers to move from the at rest configuration shown in FIG. 6 toward the deformed configuration shown in FIG. 7. As the fingers are thusly deformed, internal stresses will be formed in the fingers tending to return them to the less distended starting configuration shown in FIG. 6 As this occurs fingers 52 will exert forces on the bellows 50 which will controllably move it toward its starting configuration shown in FIG. 6. As bellows 50 moves toward its starting configuration it will exert a fluid expelling pressure on the fluid contained within the reservoir causing the fluid to be controllably forced into the rate control means of the invention via reservoir outlet 46.

During the fluid delivery step described in the preceding paragraph, fluid will flow from reservoir 44, through outlet 46, through capillary 78 of the flow control means, into fluid passageway 72 of stem 70 and finally into the delivery line 82 of the infusion means of the invention.

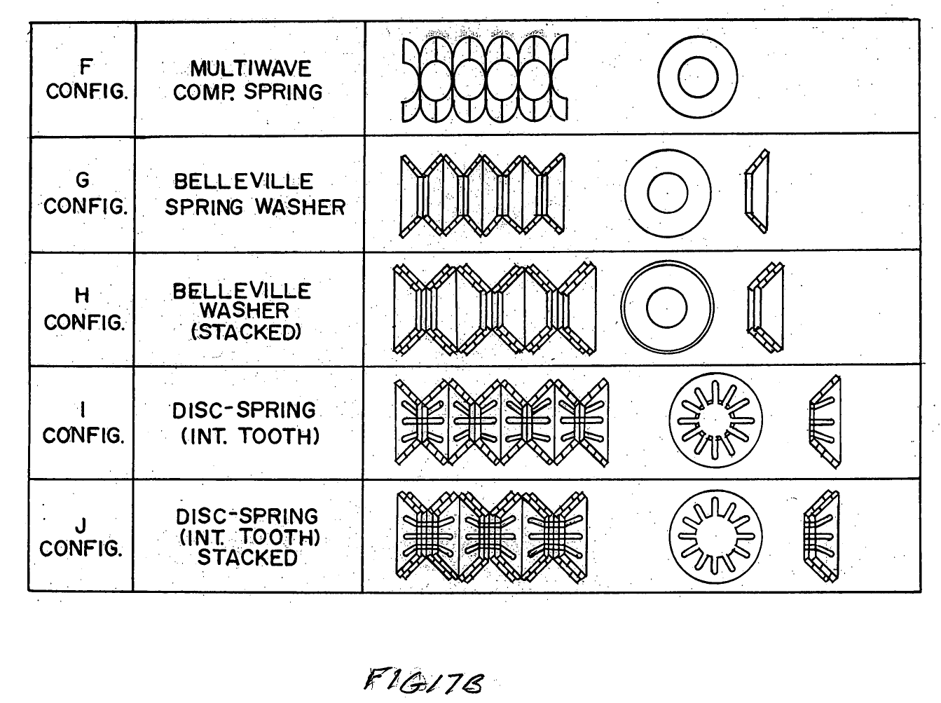

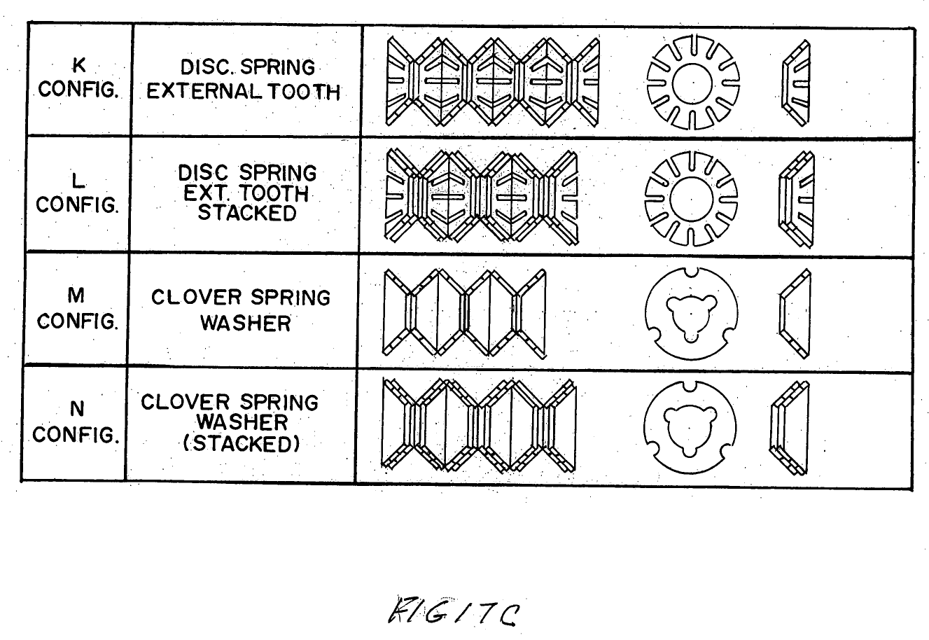

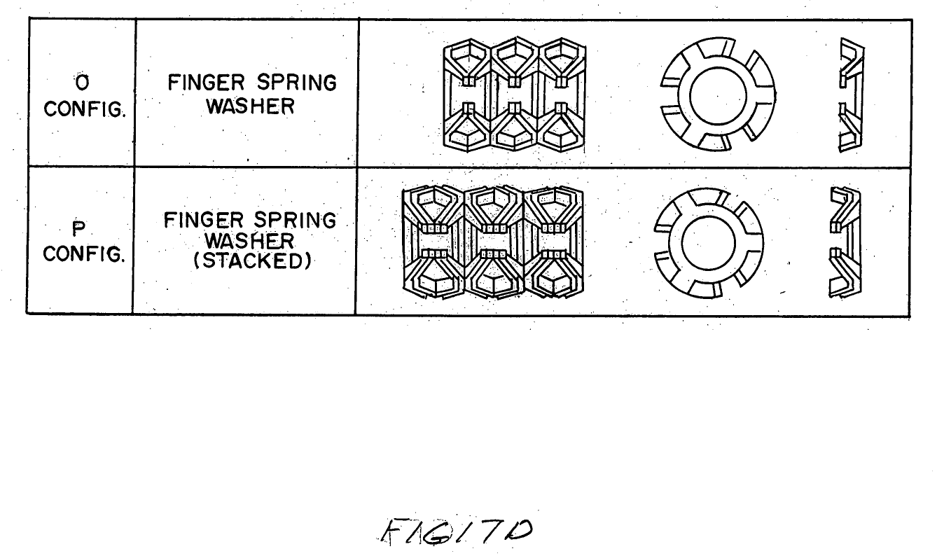

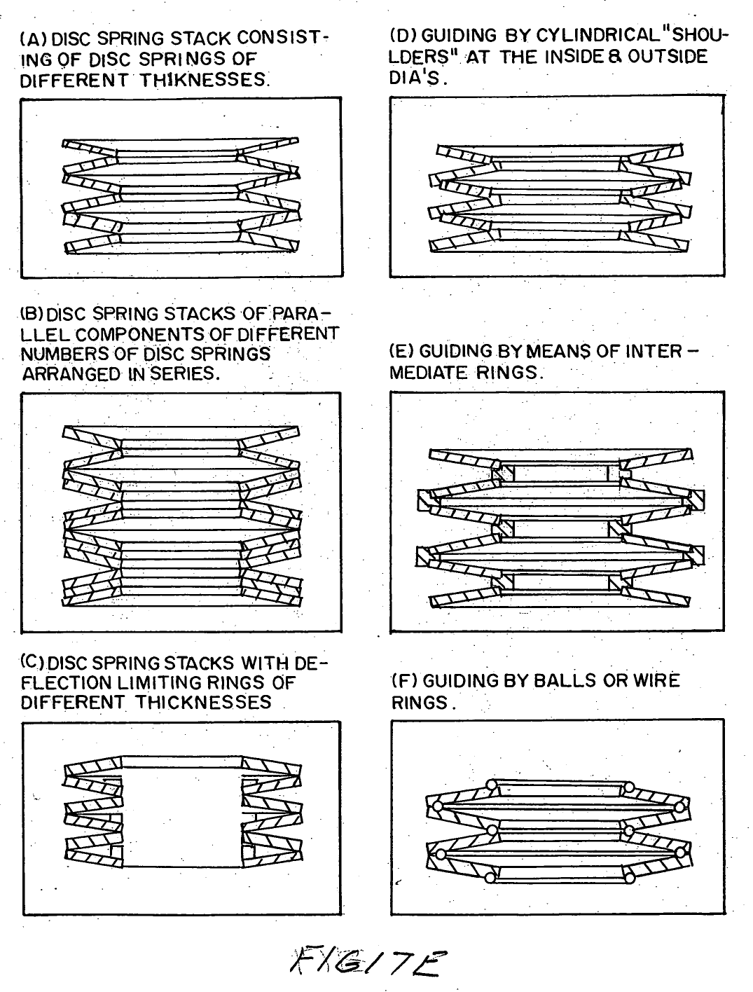

Referring to FIGS. 13, 17A, 17B, 17C 17D and 17E it is to be noted that various types of alternate spring configurations these shown are suitable for use as the stored energy source of the invention. More particularly, FIGS. 13 through 16 illustrate alternate forms of finger springs that can be used, while FIGS. 17A, 17B, 17C 17D and 17E depict a number of different types of springs that are suitable for use as the stored energy source of the invention.

In considering the various spring configurations shown in the drawings, it is to understood that, springs are unlike other machine/structure components in that they undergo significant deformation when loaded and their compliance enables them to store readily recoverable mechanical energy.

With respect to the specific spring configurations shown in FIG. 17A through 17E of the drawings, the following discussion amplifies the descriptive notations in this drawing.

Compression Springs:

Compression springs are open-wound helical springs that exert a load or force when compressed. They may be conical or taper springs, barrel or convex, concave or standard cylindrical in shape. Further, they may be wound in constant or variable pitch. The ends can be closed and ground, closed but unground, open and unground and supplied in alternate lengths. They also can include a configuration where a second compression spring of similar or different performance characteristics which can be installed inside the inside diameter of their first compression spring, i.e., a spring in a spring.

Many types of materials can be used in the manufacture with compression springs including: Commercial Wire (BS5216 HS3), Music Stainless Steel, Phosphur Bronze, Chrome Vanadium, Monel 400, Inconel 600, Inconel X750, Nimonic 90: Round wire, Square and Rectangular sections are also available. Exotic metals and their alloys with special properties can also be used for special and applications; they include such materials as beryllium copper, beryllium nickel, niobium, tantalum and titanium.

Compression springs can also be made from plastic including all thermoplastic materials used by custom spring winding service providers. Plastic springs may be used in light-to-medium duty applications for quiet and corrosion-resistant qualities.

Wave Spring:

Multiwave compression springs, an example of which is shown as “F” in FIG. 17 are readily commercially available from sources, such as the Smalley Company of Lake Zurich, Ill. As previously discussed, such springs operate as load-bearing devices. They can take up play and compensate for dimensional variations within assemblies. A virtually unlimited range of forces can be produced whereby loads built either gradually or abruptly to reach a predetermined working height. This establishes a precise spring rate in which load is proportional to deflection, and can be turned to a particular load requirement.

Typically, a wave spring will occupy an extremely small area for the amount of work it performs. The use of this product is demanded, but not limited to tight axial and radial space restraints.

Disc Springs:

Disc springs I, J, K, and L of FIG. 17 compare conically shaped annular discs (some with slotted or fingered configuration) which when loaded in the axial direction, change shape. In comparison to other types of springs, disc springs product small spring deflections under high loads.

Some examples of the disc-shaped compression springs include a single or multiple stacked Belleville washer configuration as shown in G and H of FIG. 17, and depending on the requirements of the design (flow rate over time including bolus opportunity) one or more disc springs can be used and also of alternate individual thicknesses. Alternate embodiments of the basic disc spring design in a stacked assembly can be also utilized including specialty disc springs similar to the Belleville configuration called K disc springs manufactured by Adolf Schnorr GMBH of Singelfingen, Germany, as well as others manufactured by Christian Bauer GMBH of Welzheim, Germany.

Disc springs combine high energy storage capacity with low space requirement and uniform annular loading. They can provide linear or nonlinear spring loadings with their unique ability to combine high or low forces with either high or low deflection rates. They can be preloaded and under partial compression in the design application.

All these attributes, and more, come from single-component assemblies whose nontangle features (when compared to wirewound, compression springs) make them ideal for automatic assembly procedures.

With respect to the various springs discussed in the preceding paragraphs, it is to be understood that many alternate materials can be used in the design and application of disc springs and include carbon steel, chrome vanadium steel, stainless steel, heat resistant steels, and other special alloys such as nimonic, inconel, and beryllium copper. In some special applications, plastic disc springs designs can be used.

It should be further observed that, in comparison to other types of springs, disc springs produce small spring deflections under high loads. The ability to assemble disc springs into disc spring stacks overcomes this particular limitation. When disc springs are arranged in parallel (or nested), the load increases proportionate to the number of springs in parallel, while when disc springs are arranges in series (alternately) the travel will increase in proportion to the number of springs serially arranged. These assembly methods may be combined in use.

One special feature of the disc spring is, undoubtedly, the fact that the load/deflection characteristic curve can be designed to produce a wide variety of possibilities. In addition to practically linear load/deflection characteristic curves, regressive characteristics can be achieved and even disc springs which exhibit increasing spring deflection while the corresponding disc spring load is decreasing are readily available.

Slotted disc springs present a completely different case. Slotting changes the load/deflection characteristic of the single disc spring, providing larger spring deflections for greatly reduced loads. The slotted part is actually functioning as a series of miniature cantilever arms. In some cases the stacked, slotted disc spring, as shown in the clover dome design, will also produce a non-linear, stress strain curve with a noticed flat region (force/deflection). Application and use of this type of spring operating in this region will provide a near constant force between 15% and 75% of compression.

Having now described the invention in detail in accordance with the requirements of the patent statues, those skilled in this art will have no difficulty in making changes and modifications in the individual parts or their relative assembly in order to meet specific requirements or conditions. Such changes and modifications may be made without departing from the scope and spirit of the invention, as set forth in the following claims:

1. A device for use in infusing medicinal fluid into a patient at a controlled rate comprising:
(a) a base assembly, including a base having an upper surface and a lower surface and a fluid passageway formed in said base intermediate said upper and lower surfaces, said fluid passageway having first and second ends;
(b) stored energy means for forming in conjunction with said base, a reservoir having an outlet in communication with said first end of said fluid passageway, said stored energy means comprising:
(i) an expandable bellows superimposed over said base, said expandable bellows being expanded from a first position to a second position as a result of pressure imparted by fluids introduced into said reservoir: and
(ii) at least one yieldably deformable spring member operably associated with said bellows, said spring member being yieldably deformed by movement of said expandable bellows toward said second position in a manner to establish internal stresses within said spring member, said stresses tending to move said expandable bellows toward said first position; and
(c) infusion means connected to said base assembly for infusing medicinal fluid from said fluid reservoir into the patient, said infusion means comprising a hollow cannula having an inlet end portion in communication with said fluid passageway.

2. The device as defined in claim 1 in which said stored energy means comprises a plurality of circumferentially spaced apart, a yieldably deformable spring members operably associated with said bellows.

3. The device as defined in claim 1 further including filling means connected to said base assembly for introducing fluid into said fluid reservoir.

4. The device as defined in claim 1 in which said base assembly further comprises first and second interconnected rate control plates operably associated with said base, a portion of said fluid passageway being formed in one of said first and second interconnected rate control plates.

5. The device as defined in claim 4 in which said portion of said fluid passageway formed in one of said first and second interconnected rate control plates comprises a microchannel.

6. The device as defined in claim 4 in which said microchannel is generally spiral shaped.

7. A device for use in infusing medicinal fluid into a patient at a controlled rate comprising:
(a) a base assembly, including:
(i) a base having an upper surface and a lower surface engageable with the patient and a fluid passageway formed in said base intermediate said upper and lower surfaces, said fluid passageway having first and second ends;
(ii) first and second interconnected rate control plates operably associated with said base, one of said rate control plates having a microchannel formed therein;
(iii) an expandable bellows superimposed over said base, said expandable bellows being expanded from a first position to a second position as a result of pressure imparted by fluids introduced into said reservoir; and
(iv) a plurality of yieldably deformable spring members operably associated with said allows, said spring members being yieldably deformed by movement of said expandable bellows toward said second position in a manner to establish internal stresses within said spring members, said stresses tending to move said expandable bellows toward said first position; and
(c) infusion means connected to said base assembly for infusing medicinal fluid from said fluid reservoir into the patient, said infusion means comprising a hollow cannula having an inlet end portion in communication with said microchannel.

8. The device as defined in claim 7, further including filling means connected to said base assembly for introducing fluid into said fluid reservoir, said filling means comprising a pierceable septum mounted in said base in which said

9. The device as defined in claim 7 in which said base assembly, further includes a cover superimposed over said base.

10. The device as defined in claim 7 in which said spring members comprises precision fingers springs.
